(19) United States
(12) Patent Application Publication
(10) Pub. No.: US 2005/0139487 A1
(43) Pub. Date: Jun. 30, 2005

(54) Method for the oxidative treatment of components comprised of or containing elementary silicon and/or substantially inorganic silicon compounds

(75) Inventors: Gerfried Zwicker, Itzehoe (DE); Matthias Fryda, Itzehoe (DE); Thorsten Matthee, Hohenaspe (DE)

Correspondence Address: GUDRUN E. HUCKETT DRAUDT, LONSSTR. 53, WUPPERTAL, 42289, DE

(73) Assignee: FRAUNHOFER-GESELLSCHAFT ZUR FORDERUNG DER ANGEWANDTEN FORSCHUNG E.V., MUNCHEN (DE)

(21) Appl. No.: 10/512,765

(22) PCT Filed: May 2, 2003
(86) PCT No.: PCT/EP03/04648

(51) Int. Cl. C02F 1/461; A61L 2/18

(52) U.S. Cl. 205/701; 134/2; 422/29; 422/37

(57) ABSTRACT

In a method for the treatment of an object made of silicon or an inorganic, optionally organically modified, silicon compound, an oxidizing agent is prepared by electrolysis of an aqueous solution in an electrolysis device. The anode of the electrolysis device is a silicon electrode. The anode has an overvoltage for oxygen so that upon electrolysis of water the formation of hydrogen peroxide is preferred over that of oxygen. The aqueous solution used in electrolysis contains at least one reactive component or a constituent that is converted by electrolysis of the solution into a reactive component. The object to be treated is contacted with the freshly prepared oxidizing agent. The oxidizing agent is circulated and returned into the electrolysis device to be regenerated.

### FIELD OF THE INVENTION

The present invention relates to a method with which components, prepared or constituted of, or by employing, semi-conductive silicon used in the silicon technology or other elementary, undoped or doped silicon and/or inorganic, optionally organically modified, silicon compounds such as oxides, carbides, nitrides or siloxanes, including particularly silicon wafers and other semiconductor components, can be oxidatively etched, cleaned, or treated otherwise.

### BACKGROUND OF THE INVENTION

The oxidative treatment of surfaces is of great importance in microelectronics. It is used, for example, for cleaning the surfaces of silicon wafers but also for the removal of photo resist layers (photo resists) on semiconductor disks. In some cases, contaminants comprised of foreign metals are to be removed. Suitable processes are also required for removing organic substituents or particles.

For this purpose, oxidizing solutions are used on a great scale. Such solutions are manufactured from acidic or alkaline solutions but also from water and, in particular, purest water by enrichment with hydrogen peroxide and/or ozone.

The photo resist layers that are required as masks for the structure transfer in the semiconductortechnology must be removed aftercompletion of the structuring process. Depending on the type of the resist material, suitable solvents, oxidizing chemicals, oxidizing gases or oxidizing plasma can be used for this purpose. The difficulty of this task resides in that the photo resist must be removed without leaving any traces and without attacking the underlying layers.

The oxidizing chemicals that decompose photo resist include fuming nitric acid or sulfuric acid comprising oxidizing agents. Already in 1970, W. Kern and D. Poutinen proposed in RCA Rev. 31, 187, to employ a 2:1 mixture of 98% H₂SO₄ and 30% H₂O₂ at temperatures of about 100° C.; compare also W. Kern, The Evolution of Silicon Wafer Cleaning Technology, J. Electrochem. Soc. 137, 1887 (1990). Despite the relatively high hazard potential, regularly incurred bath renewal costs, and a high demand for deionized water for rinsing the highly viscous concentrated sulfuric acid, the sulfuric acid/hydrogen peroxide mixture (SPM; “Prianha”) is employed on a large scale for microchip manufacturing by almost all manufacturers.

The actual resist removal is realized by oxidation of the organic constituents of the photo resist (for example, phenolic resins) to CO₂ and H₂O. In the SPM bath, an in-situ reaction of sulfuric acid with hydrogen peroxide to Caro's acid (peroxomonosulfuric acid) H₂SO₅ takes place, wherein the peroxomonosulfuric acid represents a very strong oxidizing agent that attacks organic compounds and decomposes them to inorganic constituents. Silicon, silicon dioxide, or silicon nitride are not attacked or hardly attacked by SPM; instead, a protective silicon dioxide layer is formed on exposed silicon and such layer may even be desirable in some cases.

Because of the high bath temperature of typically 115° C., the hydrogen peroxide will decompose rather quickly. Measures for extending the bath life include regular addition of small amounts of H₂O₂ which leads to an increasingly greater dilution with the result of a regularly required exchange of the bath or the introduction of ozone gas for continuously regenerating H₂SO₅.

In addition to immersion bath cleaning devices, SPM is used also in chemical spray cleaning devices where the hot mixture is sprayed through nozzles onto the slowly rotating silicon wafer. Depending on the type of processing, the employed chemicals are discarded or collected which can lead to improved cleaning results or reduced costs.

In the context of a general desire for cost-reduction, but also for protecting the environment, concepts have been pursued for several years with which ozone dissolved in water has taken over the role of SPM. For this purpose, an ozone/oxygen gas mixture generated in an ozone generator by means of low current electric discharge is bubbled through a water bath and ozone is dissolved in this way. The dissolved ozone oxidizes the photo resist in a way similar to SPM and decomposes it to inorganic basic constituents.

A problem is the minimal solubility of ozone in water which is within the magnitude of a few 10 ppm (mg/l). The decomposition of ozone that occurs also depends on the temperature and the pH value of the water. Photo resist removal rates of a few 100 nm/min are achievable; however, this value is below the results with hot SPM in the bath by a factor of 3-5.

Similar results are obtained when ozone is allowed to act on moist silicon disk surfaces which is realized either by rotation of the wafer while simultaneously spraying water or by generating water vapor mist and introducing ozone gas. Both methods have in common a resist removal rate of more than 500 nm/min. However, a critical aspect remains in that the removal rate is reduced in the case of photo resists that have been degraded by other processes such as plasma etching or by ion implantation.

For removal or organic substituents or contamination caused by foreign metals, for example, gold, silver, copper, cobalt, cadmium or nickel, as well as the oxidation of the wafer surface, so-called SC 2 (Standard Clean 2) is used in the semiconductor technology. It is comprised of a 1:1:6 mixture of HCl, H₂O₂, and H₂O, used at 70° C. Modern cleaning methods employ instead HF/O₃ (compare, for example, E. Bergman and S. Lagrange, Process and Environmental Benefits of HF-Ozone Cleaning Chemistry, Solid State Technology, Vol.44, 115 (2001); Y. Fukazawa et al., An HF—O₃ Aqueous Solution for Silicon Wafer Cleaning, Proc. UCPSS 1994, 267; or G.-M. Choi et al., The Role of Oxidant in HF-Based Solution for Noble Metal Removal from Substrate, Proc. UCPSS 2000, 267), HF/HCl/O₃ (compare E. J. Bergman et al., Preliminary amendment-Diffusion Cleaning Using Ozone and HF, Proc UCPSS 2000, 85) or HCl/O₃ (M. Heyns et al. Advanced Wet and Dry Cleaning Coming together for the Next Generation, Solid State Technology, Vol. 42, 37 (1999)).

A further standard solution is the so-called SC 1 (Standard Clean 1) that in the beginning was used as a mixture of one part NH₄OH, one part H₂O₂, and five parts water. In addition to dissolving organic films and complexing metals, as mentioned above in connection with the SC 1 solution, the solution effects the continuous formation and immediate dissolution of SiO₂ on the silicon surfaces. This latter process effects the release of particles from the disk surface. This process is enhanced by the formation of a negative ζ-potential of the particles as well as the surfaces at pH values greater or equal to 10. In order to reduce an etching attack of the NH₄OH and a roughening of the silicon surface that goes hand in hand, other mixture ratios with a reduced amount of ammonia have been proposed in the meantime as well as the replacement of NH₄OH with other bases such as tetramethyl ammonium hydroxide.

Because of metals dissolved in the bath, particularly iron, but also copper, the hydrogen peroxide is catalytically decomposed which can also lead to a rough surface by way of formation of oxygen bubbles and the reduction of the oxidizing agent. In this context, the H₂O₂ concentration of an SC 1 solution at 80° C. is cut in half in approximately 2.5 hours. A possible measure for compensation of the loss of H₂O₂ is the regular addition (spiking) of peroxide or bubbling ozone through the solution.

It is also known to generate a cleaning solution in that ozone is allowed to bubble through 0.1-1.0% hydrofluoric acid. The solution is suitable, for example, for cleaning silicon wafers, wherein the silicon is oxidized and the SiO₂ that has been formed is then etched off by hydrofluoric acid. Contaminants on the surface of the disk or within the oxide are dissolved.

All described methods are characterized by a high consumption of chemicals and thus incur high operational costs (for purchase and disposal) or characterized by high investment costs for the ozone generator. Methods that employ ozone dissolved in water provide photo resist removal rates that are too low, and, in the case of methods that employ ozone gas and a water film on the wafer disk surface or water vapor, the number of disks that can be treated per unit of time is limited. The service life of the baths are too short because of the high decomposition rate of the hydrogen peroxide at the required temperatures of generally more than 100° C. New baths must be prepared even though the old ones are not yet soiled or consumed. Measures for extending the service life such as regular addition of hydrogen peroxide (spiking) lead to continuously increasing dilution.

It has been known for some time to provide electrodes for manufacturing liquids that are enriched with hydrogen peroxide and/or ozone with a conductive diamond layer. This leads to a strong formation of overvoltage at the anode (up to 3 volt in comparison to 1.5 volt for platinum electrodes) and the cathode (−1.5 volt in comparison to −0.1 volt). Since the anode reaction 2 H₂O→O₂+4H⁺+4e⁻ requires a voltage of at least 1.23 volt, the anode reaction 3 H₂O→O₃+6H⁺+6e⁻ requires a voltage of at least 1.51 volt, and the anode reaction 2 H₂O→H₂O₂+2H⁺+2e⁻ requires a voltage of at least 1.78 volt, this overvoltage allows the formation of hydroxyl radicals, ozone and hydrogen peroxide while providing for a reduced decomposition of water into hydrogen and oxygen. Kurosu et al. propose in U.S. Pat. No. 6,375,827 B1 to use such electrodes for water treatment. Glesener et al. in U.S. Pat. No. 6,267,866 B1 use diamond-coated tungsten or titanium electrodes also as anodes but also primarily as a cathode for decomposition reactions, in particular, for decomposing halogenides and halogen-containing organic materials. In JP 2001-192874 A, a method for producing peroxo disulfuric acid is proposed in which electrodes of conductive silicone, silicon carbide, titanium, niobium, molybdenum, etc. coated with conductive diamond, are used as an anode. Peroxo disulfate solutions that can be used as etching solutions can be produced or regenerated according to DE 199 62 672 in that a corresponding sulfuric acid solution is electrolyzed at an anode in a two-part electrolysis cell separated by special separators, wherein the anode is comprised of valve metals that are coated with diamond that has been made conductive. With the obtained solution, copper materials, stainless steel materials and specialty metals can be treated. The limit conditions for manufacturing such solutions are disclosed in DE 199 48 184 C2. EP 0949205 A1 discloses a method for generating an aqueous solution of hydrogen peroxide and ozone of pure or ultra-pure water which solution can be used for semiconductor cleaning. As an anode, an electrode of titanium, niobium, tantalum, silicon, carbon, nickel tungsten carbide, and the like is used that is coated with conductive diamond.

### SUMMARY OF THE INVENTION

The present invention relates to a method that enables the oxidative tretament of objects manufactured/constituted or to be constituted of, or by employing, semi-conductive silicon used in the silicon technology or other elementary, undoped or doped silicon and/or inorganic, optionally organically modified, silicon compounds such as oxides, carbides, nitrides or siloxane. The method is particularly beneficial for treatment of purely inorganic surfaces. The treatment method comprises, for example, the cleaning of silicone surfaces, the removal of inorganic or organic contaminants or particles on such surfaces or stripping of photo resists that are used for surface structuring. With the method according to the invention, the above described disadvantages of the prior art are avoided.

The method according to the invention comprises the steps of:
producing an oxidizing agent by electrolysis of an aqueous solution in an electrolysis device whose anode is an electrode based on silicon on which oxygen has such an overvoltage that upon electrolysis of water the formation of hydrogen peroxide is preferred over that of oxygen, wherein the aqueous solution contains at least one reactive component and/or constituent that is converted by electrolysis of this solution into a reactive component; and
contacting the object to be treated with this freshly prepared oxidizing agent.

Preferably, the method according to the invention is designed as a circulation process in which the electrolyzed liquid is guided into a container in which contacting with the object to be treated takes place and, subsequently, is returned into the electrolysis chamber where it is refreshed by re-electrolysis.

According to a specially preferred configuration of the invention, the anode is a diamond-coated electrode.

### BRIEF DESCRIPTION OF THE DRAWINGS

FIG. 1 shows schematically a device for performing the method according to the invention.

FIG. 2 is a schematic illustration of the configuration of the electrolysis device.

### DETAILED DESCRIPTION OF THE INVENTION

The anode that is employed in the method according to the invention is such an electrode that enables a strong overvoltage of oxygen. As mentioned above, the oxygen formation at a “normal” anode occurs already at 1.23 volt, the formation of ozone on the other hand however at a voltage of 1.51 volt, and that of hydrogen peroxide even at 1.78 volt. When an anode with high overvoltage for oxygen is selected, the formation of the species ozone and hydrogen peroxide is preferred, at very high voltages hydroxyl radicals are even formed, while the formation of oxygen is greatly reduced or substantially suppressed. According to the invention, the phrases “an electrode based on silicon on which the oxygen has a high overvoltage” and “an electrode based on silicon on which the oxygen has such an overvoltage that upon electrolysis of water the formation of hydrogen peroxide is preferred over that of oxygen” are meant to describe these circumstances. When electrolyzing water with such an electrode, the generation of ozone and/or hydrogen peroxide is preferred in comparison to that of oxygen; more ozone and/or hydrogen oxide molecules then oxygen molecules are produced.

As mentioned before, measures are known with which such overvoltages can be obtained. One such measure is, for example, coating of the electrode surface with a conductive diamond layer. In regard to this, reference is being had to the prior art mentioned in the introduction.

According to the invention, the solution to be electrolyzed contains in addition to water also at least one reactive component and/or a constituent which upon electrolysis of this solution is converted directly or by means of the species generated by electrolysis of this solution into a reactive component. Oxidizing agents or complex forming agents but also strong acids can be used as an already reactive component. Examples are hydrofluoric acid, hypochlorite or ammonia that, in alkaline environment, forms stable amine complexes with a series of metals, for example, copper. Also, high proton concentrations upon use of strong, primarily inorganic, acids are an example for a reactive component. The phrase “constituent that is converted upon electrolysis of this solution directly or with the species generated by electrolysis in the solution into a reactive component” is to be understood as meaning compounds or ions which are oxidized directly at the anode or react with ozone or hydrogen peroxide to form a usually strong oxidizing agent. As an example sulfuric acid should be mentioned whose sulfate ions are oxidized in the method according to the invention at the anode to peroxo disulfate. Also, hydrochloric acid can be used wherein hydrochloric acid can function with regard to its properties as a reactive component, i.e., as a strong acid when used in appropriate concentration, but also as a constituent that can be converted upon electrolysis of this solution directly or with the species generated by electrolysis of this solution into a reactive component, because, depending on the process control and the employed voltage, the anion chloride can form atomic chlorine or hypochlorite, for example, by reaction with hydrogen peroxide, at the anode.

By means of the method according to the invention, the highly aggressive oxidizing agent ozone and/or hydrogen peroxide is generated in-situ in the liquid that is being electrolyzed. In combination with the reactive component or components, it is thus possible to effectively clean or free from layers to be removed surfaces that are needed in silicon technology or the like. An external ozone generation or hydrogen peroxide generation is not required when in the method, as is preferred, the electrolysis chamber is combined integrally with the treatment vessel.

The constituents of the solution to be electrolyzed are selected in a suitable manner as a function of the objectives.

In a first preferred configuration of the invention, the constituent that is converted to a reactive component by electrolysis is sulfuric acid or a sulfate; this configuration is suitable inter alia for stripping off photo resist. The electrolysis of sulfuric acid or sulfate with an anode that enables the generation of a suitable overvoltage leads to formation of peroxo disulfuric acid and/or peroxo disulfate.

In the past, the so-called Caro's acid H₂SO₅ has been used for stripping photo resist which acid is formed in the so-called SPM solution, as explained in the introduction. Even though it is generally known in the literature that the peroxomonosulfuric acid is in an equilibrium reaction with the peroxo disulfuric acid, the latter has never been used for this purpose up to now, even though it is commercially available. A reason for this could be the contamination with metals of commercially available peroxo disulfuric acid because it is produced by employing platinum electrodes and additives. Such contaminants are not tolerable for chemicals that are provided for semiconductor or silicon technology.

According to the invention, the peroxo disulfuric acid is produced by employing electrodes based on silicon so that no contamination by foreign metals can be introduced as a result of the electrolysis step. Even when the coating of the electrode is not tight, but has small holes, no metal ions can be removed from the electrode material that would impair the purity of the chemicals as contamination.

In the method according to the invention, the generation of the peroxo disulfuric acid takes place even at minimal concentrations of the electrolyte liquid at high conversion efficiency. With regard to limit conditions for its preparation, reference is being had, for example, to DE 199 48 184 C2 whose disclosure is to be understood as being incorporated fully into the instant application. Suitable for the method according to the invention are sulfuric acid or sulfate concentrations across a wide range that can be from 1% to 50%. In particular, relatively minimal concentrations of sulfuric acid are beneficial because the desired effect occurs usually already at such concentrations and the costs for the purchase and disposal of chemicals can be kept low. Preferably, during electrolysis the cathode chamber is separated by an ion-conductive membrane from the anode chamber in order to prevent decomposition of the freshly formed peroxo disulfate at the cathode.

In a second preferred configuration of the invention, hydrofluoric acid is selected as a reactive component. The electrolysis of the dilute hydrofluoric acid with the electrodes to be used according to the invention leads, depending on the process control (supplied voltage) to the formation of ozone and/or H₂O₂. The ozone is dissolved in the bath and is available as an oxidizing agent. Mixtures of HF and ozone are very aggressive and well-suited in order to remove, for example, copper and other metals that otherwise can be removed only with great difficulty from silicone surfaces and can be hardly removed even with the above mentioned peroxo disulfuric acid. Even though HF/O₃ mixtures have a potentially higher oxidation/reduction potential than HF/H₂O₂ solutions, the efficiency of the latter is optionally still higher because the ozone is soluble only to a limited extent while significantly higher concentrations of peroxide can be obtained in the solutions.

The method according to the invention with electrolysis of hydrofluoric acid is suitable, for example, also for the manufacture of silicon wafers. Moreover, it can be used also to remove particles or organic contaminants from silicon surfaces, inter alia, also from already structured wafers. Already very small concentrations of hydrofluoric acid, for example, 0.01 to 1% by weight, preferably approximately 0.1% by weight, can provide excellent results.

In a third preferred configuration of the invention, hydrochloric acid or chloride is selected as a constituent that is converted upon electrolysis—directly and/or indirectly—into a reactive component. As mentioned before, electrolysis can lead to the formation of reactive chlorine at the anode.

According to a fourth preferred embodiment of the invention, the reactive component is ammonia. This variant is particularly well suitable for cleaning very finely structured wafer disks because at a pH of more than 7 the disk and possibly adhering very fine particles reach identical ζ-potential so that the particles can be removed more easily. Moreover, in this configuration copper can particularly well form a complex with the oxidizing agent and can thus be removed easily. Minimal concentrations of ammonia, for example, in the range of 0.2 to 1 mol-%, in the solution to be electrolyzed are well-suited for cleaning silicon wafers in this configuration. In this way, a higher selectivity is achieved for the cleaning action while the silicon surface is not attacked.

For the method according to the invention, as mentioned above, an anode is used on which the oxygen has such an overvoltage that upon electrolysis of water the formation of hydrogen peroxide is preferred over that of oxygen. Such an overvoltage can be obtained particularly well by coating the electrode with conductive diamond. In this regard, reference is being had to the prior art mentioned in the introduction. Well suited are diamond films having a thickness of approximately 1-20 μm that have been made conductive by doping with boron or nitrogen.

The diamond film is deposited on an electrode body of silicon (for example, a silicon wafer) or applied in other ways, wherein the electrode body itself preferably has been made conductive. This can also be realized, for example, by doping with boron. The selection of silicon as electrode material prevents contamination of the generated solution by materials foreign to the process even if the diamond coating should have small holes. Low impedance silicon is etch-resistant and is not attacked, for example, by peroxo disulfuric acid. It is however still preferred that the diamond layer on the electrode should be as tight as possible in order to prevent the OH radicals that are produced at high current densities from attacking the silicon surface.

In the process according to the present invention, not only must the anode have the property of allowing an overvoltage but the cathode can also have this property. It can therefore be designed in the same way as the anode.

The rear contacting of the electrodes in the case of the anode as well as the cathode can be realized by tantalum or other metals, wherein the contacting surface must be protected in a suitable way against the surrounding chemicals, for example, must be encapsulated, so that no metal ions or similar materials can reach the electrolysis bath.

It is expedient to provide stacking of the electrodes in order to obtain electrolysis cells that are series-connected. This provides a higher oxidizing agent formation rate. In this case, only the first and last electrodes must be contacted while the intermediately positioned electrodes function on one side as an anode and on the other side as a cathode.

In all cases, an ion-conductive membrane or any other suitable blocking arrangement can be provided in the electrolysis chamber for separating the cathode chamber from the anode chamber. This is particularly beneficial when, as in the case of electrolysis of sulfuric acid, the product or products formed at the anode would be decomposed again at the cathode.

As disclosed in the published references DE 19948 184 C2 and DE 299 16 126 U1, it is particularly favorable to operate the electrolysis device at a voltage that is as high as possible in order to obtain a proportion of H₂O₂ as high as possible, which voltage however should be below the voltage at which oxygen formation at the anode will noticeably occur. The voltage between anode and cathode can be adjusted if possible such that it is less than 25%, preferably less than 15%, below the voltage at which oxygen formation at the anode begins. In this way, highly reactive oxidizing agents can be produced without the efficiency being noticeably reduced by the generation of oxygen.

The advantages that can be obtained with the invention in comparison to conventional methods that are usually employed in practice for the oxidative treatment of surfaces are numerous:

When employing the method according to the invention as a replacement for the treatment methods with hydrogen peroxide, significant cost reductions can be realized, uncontrolled bath dilution can be prevented, and longer service life of the baths can be achieved. By employing significantly less acid by means of reduced concentrations and longer periods of use of the baths and the elimination of supplied peroxide the environmental loads are reduced.

When replacing the sulfuric acid/hydrogen peroxide mixture with the method according to the invention, the concentration of the employed sulfuric acid can be significantly reduced, a significant reduction of costs can be realized, and manipulation as a result of reduced viscosity and easier rinsing of the sulfuric acid with reduction of the hazard potential can be prevented.

Moreover, the separate generation of ozone is no longer required so that a reduced space requirement, elimination of installation costs, and lowering of energy costs and material costs will result.

The oxidizing agent to be used according to the invention can be contacted with the object to be treated outside of the electrolysis bath in which it has been generated by electrolysis. Contacting can be realized in any suitable way. For example, the method according to the invention is suitable for use in immersion bath devices as well as chemical spraying devices, wherein, for example, individual wafers or wafer stacks can be treated. The treatment can also be based on sound generation. The temperature, depending on the application, can be selected in an optimal range and is in general between 0° C. and 170° C.

The method according to the invention is configured preferably as a circulation process wherein the oxidizing agent is generated in an electrolysis device and is subsequently guided into a treatment chamber where the objects to be treated are contacted with it, for example, by spraying or immersion. Subsequently, the oxidizing agent, optionally after a cleaning step such as filtration, is returned into the electrolysis device where it is refreshed by undergoing electrolysis again. If needed, a partial stream can be removed from the circulating stream and fresh solution can be added.

The oxidizing solution is generated in the electrolysis device especially preferred at a temperature that will level out at an equilibrium temperature of approximately 50° C. Especially preferred are also values of 40-60° C. The treatment in the treatment chamber can be realized in particular for the resist stripping method by employing oxidized sulfuric acid but also in other cases at approximately 100-160° C., preferably at or above 130° C., and particularly preferred at approximately 140-150° C.

In order to realize the aforementioned temperatures or similar temperatures, the oxidizing solution on its path to the treatment chamber, for example, shortly before entering the chamber, can be heated in a suitable way to higher temperatures, for example, by heat exchangers such as a flow heater. The treatment chamber can be designed, for example, as an immersion chamber or a spraying chamber. During treatment, the solution will cool down by itself when the wafer in the treatment chamber has not been pre-heated. With a suitable conduit system, a cooling to not more than 60° C. until reentering the electrolysis device can be achieved, optionally also by heat exchange or the like. Before the oxidizing solution enters the electrolysis device again, a cleaning step, for example, a filtration step, can be carried out.

Alternatively, the objects to be treated can be pre-heated. For example, in the treatment of e.g. silicon wafers, the wafers can be kept floating by means of a heated gas stream supplied from the rear and at the same time can be heated to a suitable temperature, for example, to the aforementioned treatment temperature. Alternatively, the wafers can be heated by radiant heat, for example, infrared heating. Finally, it is also possible to place the wafer onto a heated support and to heat it by contact heat. The heated objects can then be treated by spraying the cooler oxidizing solution that has not been heated or not completely heated onto the objects. The solution will be heated to the optimal treatment temperature by contacting the object to be treated. Optionally, reaching the temperature conditions can be assisted by additional heating of the solution before entering the treatment chamber.

Alternatively, the entire circulation process can be carried out at a uniform temperature, for example, at approximately 50° C.

In the following, the invention will be explained in more detail by means of examples.

### EXAMPLE 1

Two electrodes coated with conductive diamond were immersed in sulfuric acid of a concentration of approximately 33% in a beaker. The base of the electrodes was comprised of p-conductive silicon doped with boron and having a conductivity that corresponds to a resistivity of 5 mΩcm wherein the silicon was coated with a diamond film of a thickness of approximately 10 μm, done by means of so-called hot filament CVD. The diamond film was doped also with boron and accordingly was also p-conductive. The electrodes were contacted at the rear. The cathode was separated by a conductive membrane (Nafion® membrane) from the anode in order to prevent decomposition of the freshly formed peroxo disulfates at the cathodeode. The electrode surface was 160 cm², the supplied current 16 A, i.e., the working current density was 100 mA/cm². After an enrichment period of 20 hours, corresponding to approximately 11 Faraday, the solution was colored deeply yellow and heated to an equilibrium temperature with the surroundings of approximately 50° C.

### EXAMPLE 2

Silicon wafers were coated with conventional photo resist (resin based on novolak, i.e., on the basis of phenols forming chains by means of CH₂ groups, with diazonaphtho quinone as a photo active component that still contained residual amounts of the solvent propylene glycol monomethyl ether (PGME)). A portion of the coated wafers was subjected to conventional processes as they may be required for manufacturing microchips. One wafer was exposed to a plasma etching process. The photo resist of another wafer was irradiated with deep UV. Third and fourth wafers were exposed to ion implantation of arsenic and boron, respectively, at high doses in the range of 8×10¹⁵/cm² and ion energies in the range of 40-80 keV. All of these processes lead to the resist being damaged. The ion implantation moreover leads to a new crosslinking of the resist which causes incrustation of the resist surface (carbonizing). Moreover, exposed silicon dioxide is removed by sputtering, is deposited on the resist and leads to incrustation. Such resists can be stripped only with great difficulty even with Caro's acid.

The wafers coated with resist and partially treated further were heated on a heating plate to 100-150° C., and small amounts (≦0.5 ml) of the peroxo disulfuric acid prepared as described in Example 1 were dripped thereon and heated in a short period of time to the wafer temperature. In doing so, the resist layers were etched.

The process of resist removal was performed partially by etching, i.e., removal, partially by sub-etching, i.e., peeling. Macroscopically visible peeled-off flakes were removed from the wafer and placed into the beaker with the oxidizing solution that was at equilibrium temperature. After approximately five minutes the flakes were completed dissolved.

The resists themselves were removed after approximately 5-8 minutes at 140-150° C.

### EXAMPLE 3

In order to simulate a spin etching process, wherein new solution is dripped continuously onto a wafer that rotates not very quickly and is spun off by centrifugal force, 0.25 ml of the oxidizing solution, prepared according to Example 1, was applied at intervals of one minute at a temperature of 50° C. onto the resist-coated wafer that had been subjected to As implantation in accordance with the parameters described in Example 2 and was positioned on a heating plate at a temperature of approximately 100-150° C. After six minutes, the wetted portion of the wafer was free of resist.

### EXAMPLE 4

Example 3 was repeated with an oxidizing agent prepared according to Example 1 with the modification that it contained additionally fluoride ions in a concentration of 10 ppm.

The removal of the resist occurred even faster than in Example 3.

### EXAMPLE 5

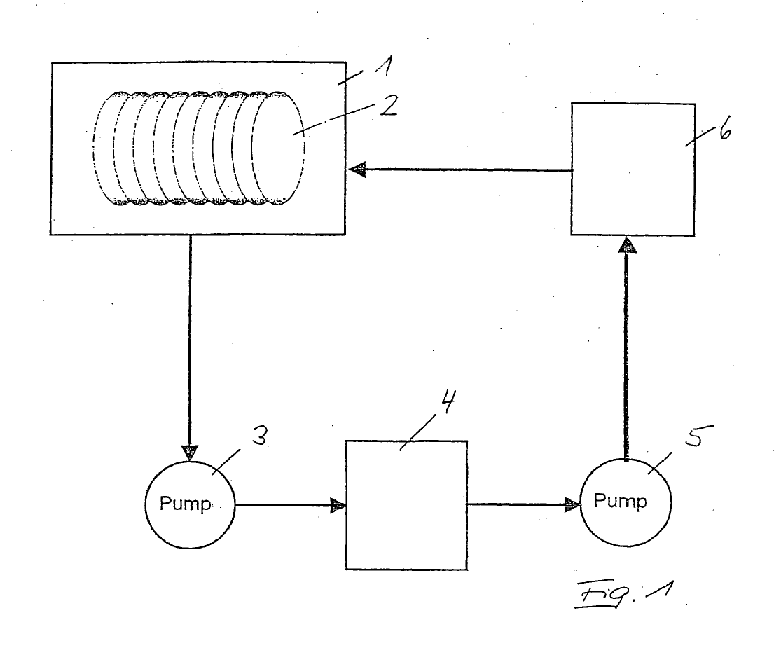

FIG. 1 shows an embodiment of a device according to the invention for oxidative treatment of surfaces that can be used inter alia for the method according to the present invention resulting chemically from the combination of Examples 1 and 2, i.e., for removal of photo resist from wafer surfaces.

An appropriate treatment chamber 1 receives a stack of silicon wafers 2 and is filled with an oxidizing solution so that the silicon wafers are immersed completely in the oxidizing solution. The oxidizing liquid is pumped off at the bottom of the treatment device 1 by a first pump 3 and is guided into an electrochemical device 4. The oxidizing liquid that has been refreshed within the electrolysis device 4 is forced by a second pump 5 into a conditioning container 6 and forced from there into the treatment device 1. In the conditioning container 6, the oxidizing solution is essentially heated to operating temperature and/or filteed.

The oxidizing solution is based on a sulfuric acid contents that can be as provided in Example 1 and should be in particular less than 50%. It can also be less than 25%. As a result of the electrochemical treatment in the electrolysis device, hydroxyl radicals are formed from the water contents of the sulfuric acid; they provide for an efficient production of peroxo disulfuric acid even from the dilute sulfuric acid. As byproducts, further oxidizing agents such as ozone, peroxomonosulfuric acid and hydrogen peroxide can be performed.

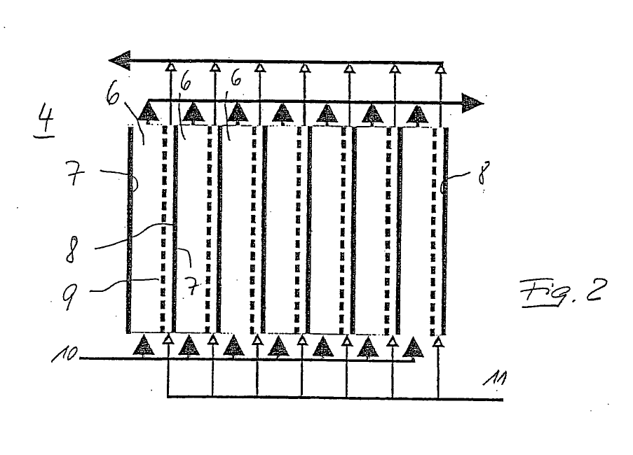

A specific configuration of the electrolysis device 4 is illustrated in FIG. 2. It is comprised of several cells 6 that each have an anode 7 and a cathode 8. Since the anode 7 as well as the cathode 8 each are in the form of a diamond-coated electrode, between anode 7 and cathode 8 an ion-exchange membrane 9 is provided, respectively, as a blocking device for preventing transport of material.

The cells 6 are connected in series so that only the outer anode 7 and the outer cathode 8 must be contacted and supplied with a voltage.

The central electrodes are expediently configured as bipolar electrodes with doped diamond coatings on both sides so that the same electrode can act as a cathode on one side for a cell 6 and as an anode 7 on the other side for the neighboring cell 6.

In the configuration according to FIG. 2, the oxidizing solution 2 flows through the cells 6 divided uniformly, in particular at the anode side of the ion exchange membrane 9.

The oxidizing solution is generated by electrolysis in the electrolysis device 4 by reaction with the dilute sulfuric acid and is enriched with the required oxidizing agents.

At the cathode side of the ion exchange membrane 9 any suitable electrolyte 11 is added to the cells as a catholyte and is also transported in circulation. The catholyte is provided only for the ion exchange action and can therefore remain unaffected by the electrolytical processes.

1-19. (canceled)

20. A method for the treatment of an object that, partially or entirely, is comprised or is constituted or is to be constituted of silicon and/or inorganic, optionally organically modified, silicon compounds, the method comprising the steps of:
a) preparing an oxidizing agent by electrolysis of an aqueous solution in an electrolysis device having an anode that is an electrode comprised of silicon, wherein the anode is configured such that oxygen has an overvoltage at the anode so that upon electrolysis of water of the aqueous solution formation of hydrogen peroxide is preferred over formation of oxygen, wherein the aqueous solution comprises at least one reactive component and/or a constituent that is converted by electrolysis of the aqueous solution into the at least one reactive component; and
b) contacting the object to be treated with the freshly prepared oxidizing agent of step a).

21. The method according to claim 20, further comprising the step of circulating the oxidizing agent by returning the oxidizing agent after the step b) into the electrolysis device and regenerating the oxidizing agent in the electrolysis device.

22. The method according to claim 20, wherein in the step a) the oxidizing agent is produced at a temperature from 40-60° C. and wherein in the step b) at least one of the oxidizing agent and the object has a temperature of 100-160° C.

23. The method according to claim 22, wherein in the step a) the oxidizing agent is produced at a temperature of approximately 50° C. and wherein in the step b) the temperature is 140-150° C.

24. The method according to claim 20, wherein the step b) is carried out in a treatment chamber, the method further comprising the step of heating the oxidizing agent on a path into the treatment chamber.

25. The method according to claim 20, wherein the step b) is carried out in a treatment chamber, the method further comprising the step of heating the object in the treatment chamber.

26. The method according to claim 25, wherein in the step of heating the object a heated gas stream is flowed against the object.

27. The method according to claim 25, wherein in the step of heating the object radiant heat is employed.

28. The method according to claim 25, wherein in the step of heating the object contact heat provided by a solid support is employed.

29. The method according to claim 20, wherein the at least one reactive component is selected from the group consisting of sulfate ions, fluoride ions, chloride ions, and ammonia.

30. The method according to claim 20, wherein the at least one reactive component are sulfate ions and fluoride ions.

31. The method according to claim 20, wherein the electrolysis device comprises a cathode that is an electrode comprised of silicon, wherein the cathode is configured such that oxygen has an overvoltage at the cathode so that upon electrolysis of water formation of hydrogen peroxide is preferred over formation of oxygen.

32. The method according to claim 31, wherein the cathode is provided with a coating of conductive diamond.

33. The method according to claim 20, wherein the anode is provided with a coating of conductive diamond.

34. The method according to claim 20, wherein the aqueous solution to be electrolyzed has a pH value of maximally 3.5

35. The method according to claim 34, wherein the aqueous solution to be electrolyzed has a pH value of maximally 2.

36. The method according to claim 35, wherein aqueous the solution to be electrolyzed has a pH value of maximally 1.

37. The method according to claim 20, wherein the solution to be electrolyzed has a pH value greater 7.

38. The method according to claim 37, wherein the solution to be electrolyzed has a pH value equal to or greater than 10.

39. The method according to claim 20, wherein the aqueous solution to be electrolyzed contains sulfate ions as the at least one reactive component and wherein during electrolysis a voltage is supplied that leads to the formation of peroxo disulfate ions.

40. The method according to claim 39, wherein the aqueous solution to be electrolyzed contains additionally fluoride ions as the at least one reactive component.
